(19) United States
(12) Patent Application Publication
(10) Pub. No.: US 2004/0175004 A1
(43) Pub. Date: Sep. 9, 2004

(54) Error signal processing to reduce spectral overlap in an active noise control system

(76) Inventor: Manish Vaishya, Rochester Hills, MI (US)

Correspondence Address: CARLSON, GASKEY & OLDS, P.C., 400 WEST MAPLE ROAD, SUITE 350, BIRMINGHAM, MI, 48009, US

(21) Appl. No.: 10/793,541

(22) Filed: Mar. 4, 2004

Related U.S. Application Data
(60) Provisional application No. 60/452,672, filed on Mar. 7, 2003.

(51) Int. Cl. A61F 11/06; H03B 29/00; G10K 11/16

(52) U.S. Cl. 381/71.9; 381/71.11

(57) ABSTRACT

An active noise control system (20) includes a controller (34) having an error signal processing module (38). The controller (34) updates a value of an adaptive filter (40) based upon an averaged error signal (56). A convergence factor (58) used in a least mean square update equation (60) can be selected independent of an averaging parameter τ_{E}, which allows for faster convergence without the otherwise associated instability.

### CROSS REFERENCE TO RELATED APPLICATIONS

This application claims priority to U.S. Provisional Application No. 60/452,672 which was filed on Mar. 7, 2003.

### BACKGROUND OF THE INVENTION

Active noise control systems typically include a controller and a speaker for generating an attenuation signal or sound to at least partially attenuate or cancel noise from a source, such as a vehicle engine. In vehicle applications, for example, engine noises can be conducted through the air induction system associated with the engine. For example, engine noise may travel through the air induction system and emanate out of the mouth of the air intake such that the noises are noticeable in the passenger compartment of the vehicle. There are other situations where noise control is applied.

The attenuation signal or sound from the speaker is typically out of phase with the noise from the noise source and combines with the noise. The result is a reduced noise, which in the case of a vehicle application results in less noise transmission into the passenger compartment.

Typical active noise control systems utilize a microphone that detects an error signal indicating the amount of noise at the point of interest. The error signal is processed to adjust an adaptive filter to produce an ideal control output for generating the attenuation signal. Most active noise systems use a least mean square (LMS) approach to update the coefficients of the adaptive filter. LMS approaches provide a fairly good compromise between computation speed and accuracy by closely approximating the ideally required attenuation signal. The update value for the adaptive filter comprises the amount of error such that a larger error will provide a faster update.

In multi-channel control systems, which are typical of the multiple harmonics of engine noise, the error signal is derived from the analog signal measured through a transducer such as a microphone. With a typical LMS approach, a single equation is used to decompose the error signal into components corresponding to each channel of control and to conduct the control update. Having these two functions in a single LMS equation provides less than optimal performance for certain channels. Additionally, there are periodic variations in the error signal, which causes the update terms to fluctuate unnecessarily.

There is a need for an improved ability to process an error signal for updating an adaptive filter value. This invention addresses that need while avoiding the shortcomings and drawbacks of previous attempts.

### SUMMARY OF THE INVENTION

In general terms, this invention is an error signal processing technique for use in an active noise control system that reduces spectral overlap in an adaptive filter update procedure.

One example method for processing an error signal in an active noise control system includes determining an average value of the error signal. Once the average value is determined, an adaptive filter value is updated using the average error signal value. By separating out the averaging of the error signal and the updating of the adaptive filter value, this approach prevents certain instabilities in the system, allows for faster convergence and better noise control becomes possible.

In one example, the filter value is updated using a least mean square update equation. A convergence factor used in the least mean square update equation is chosen to minimize the number of cycles required for convergence. By first determining an average value of the error signal, the convergence factor can be selected independent of the need for avoiding instabilities otherwise caused by fluctuations in the error signal. The average error signal value eliminates such instabilities.

An example active noise control system includes a controller that determines an average value of an error signal. The controller then updates an adaptive filter value using the determined average error signal.

The various features and advantages of this invention will become apparent to those skilled in the art from the following detailed description of the currently preferred embodiment. The drawings that accompany the detailed description can be briefly described as follows.

### BRIEF DESCRIPTION OF THE DRAWINGS

FIG. 1 schematically illustrates an active noise control system designed according to an embodiment of this invention, which is associated with an automotive vehicle.

FIG. 2 schematically illustrates selected additional details of the embodiment of FIG. 1.

FIG. 3 schematically illustrates an error signal processing module of the controller of FIG. 2.

FIG. 4 graphically illustrates an engine noise signal.

FIG. 5A graphically illustrates an example error signal resulting from processing used in one example embodiment of this invention.

FIG. 5B graphically illustrates a corresponding error signal resulting from a prior art approach.

FIG. 6A graphically illustrates an error product term resulting from processing used in an embodiment of this invention.

FIG. 6B graphically illustrates a corresponding error product term from the prior art.

### DETAILED DESCRIPTION OF THE PREFERRED EMBODIMENT

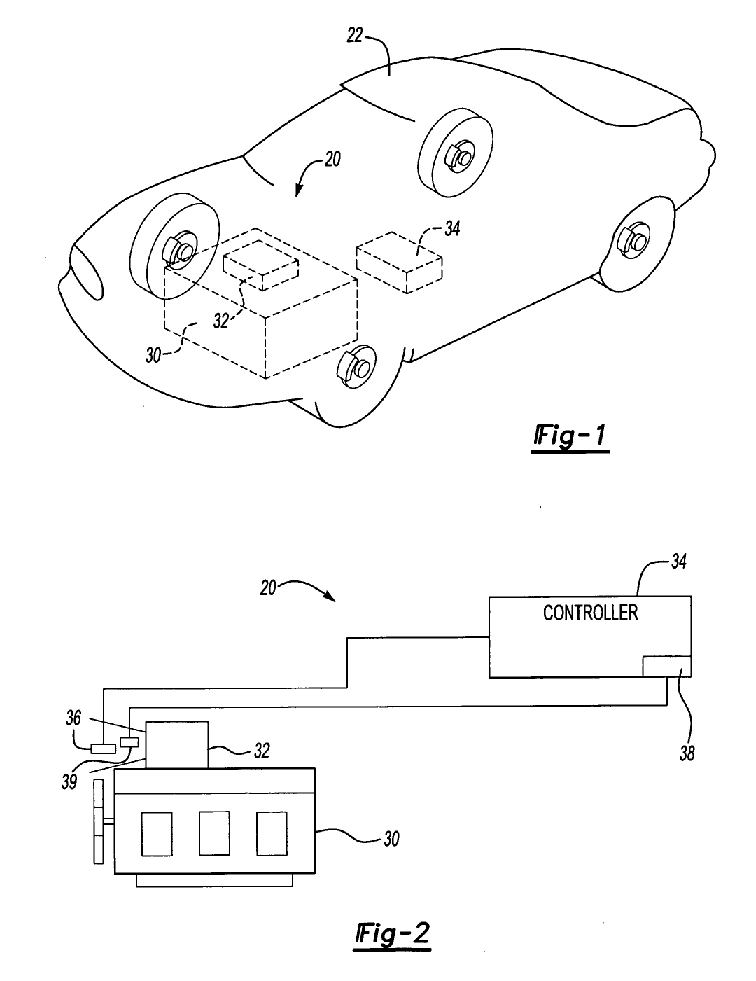

FIG. 1 schematically illustrates an active noise control system 20. The example embodiment is shown associated with a vehicle 22. This invention is not limited to active noise control for vehicle applications. Other active noise control systems will benefit from the techniques of an embodiment of this invention.

As schematically shown in FIGS. 1 and 2, the vehicle 22 includes an engine 30 having an air intake manifold 32. The active noise control system 20 includes a controller 34 that includes a combination of hardware and software that operates the noise control system in a generally known manner.

The controller 34 drives a speaker 36 associated with the air intake manifold 32 to generate a noise attenuation signal to modify or cancel out noise caused by operation of the engine 30.

The controller 34 in this example includes an error signal processing module 38 that processes an error signal (i.e., a feedback signal from a microphone 39) in a unique manner and updates an adaptive filter value used to achieve the control signal.

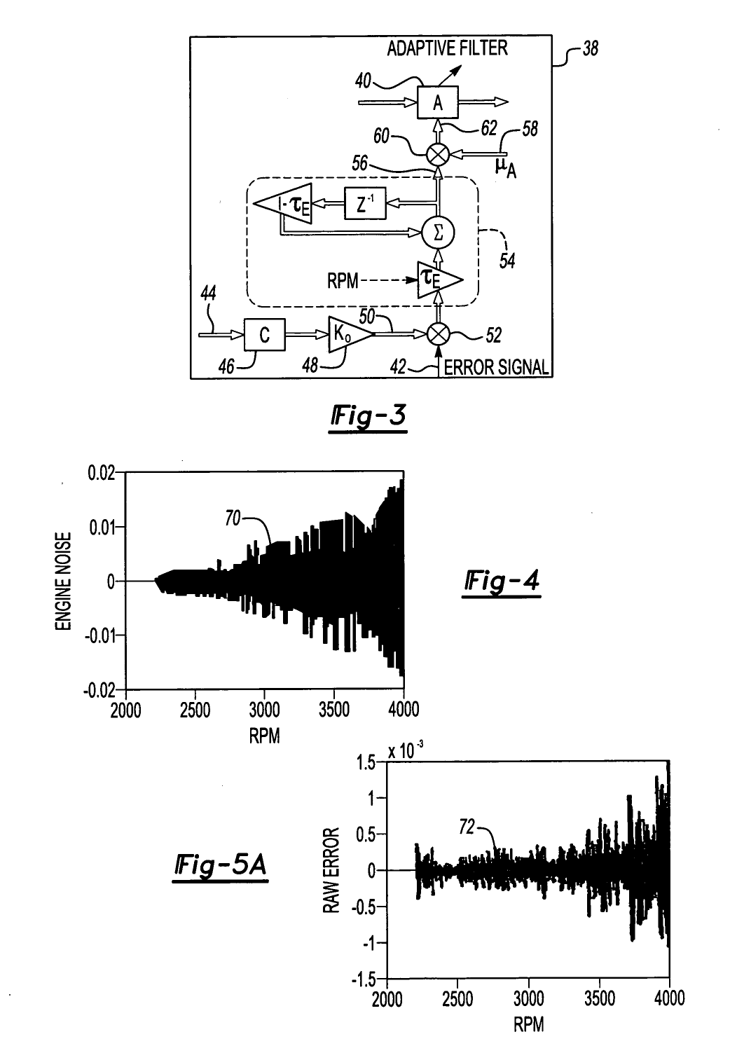

Referring to FIG. 3, the error signal processing module 38 is schematically shown as a portion of the controller 34. An adaptive filter 40 has a filter value that is used for generating an attenuation signal to achieve a desired amount of noise control (i.e., cancellation). The error signal processing module 38 receives an error signal 42 and a digitally generated reference signal 44 that is processed by filters 46 and 48 in a known manner to generate an error multiplying factor 50. The error signal 42 and the multiplying factor 50 are multiplied using a multiplier 52.

An averaging module 54 determines an average value of the multiplied error signal 42. The averaging module 38 utilizes an averaging parameter τ_{E} and generates an error product term 56 that retains the DC component of the error signal 42. The averaging module 54 distils the specific order to be controlled from the error signal 42. It is known that error signals can include periodic variations because of the nature of the pulsating signals. The averaging performed by the averaging module 54 eliminates such variations. The error product term signal 56 is then combined with a convergence factor 58 using a least mean square (LMS) equation 60. A resulting update signal 62 provides an update for the adaptive filter 40, which is then used in a known manner to modify the control signal for generating the attenuation signal from the speaker 36.

In the illustrated example, the averaging module 54 utilizes single pole averaging techniques. Other examples include using accumulating averages or running averages. Those skilled in the art who have the benefit of this description will be able to select an appropriate averaging technique to meet the needs of their particular situation. The averaging technique should be selected to provide a good power estimate in each of the channels of control.

The averaging module 54 effectively carries out orthogonal cancellation of tones, which are part of the error signal having different frequencies from the relevant component of the error signal.

The averaging parameter τ_{E} preferably is chosen to capture between about one and about two half-cycles of the lowest significant frequency. In one example, τ_{E} is determined by:

τ_{E}≈fₘᵢₙ/f_{sampling},

where fₘᵢₙ is derived from the noise source operation (i.e., engine RPM in the illustrated example) and f_{sampling} is a number selected based upon system parameters such as the bandwidth of the control. The averaging parameter τ_{E} can be adjusted to minimize spectral overlap. Additionally, the value of τ_{E} can be automatically changed responsive to a change in fₘᵢₙ, which changes when the operation of the noise source changes (i.e., a change in engine rpm's in the illustrated example). Those skilled in the art who have the benefit of description will be able to select an appropriate f_{sampling} value or table of values to meet the needs of their particular situation.

With the disclosed approach, the selection of τ_{E} provides great flexibility and allows for reducing any fluctuations or variations from the feedback error signal before that is utilized for updating the adaptive filter value. By separating out this portion of the error processing from a LMS update technique, the disclosed embodiment provides the ability to choose a LMS convergence factor μ without any restraints otherwise associated with instabilities stemming from fluctuations in the error signal. Accordingly, the value of the convergence factor μ can be selected to provide convergence as fast as desired to minimize the number of cycles of an LMS update equation without concern that the higher convergence factor value will introduce instabilities into the system.

In one example, the error product signal 56 is described as eX_{c} where e=Σe_{n,cos} cos(nωt)+e_{n,sin} sin(nωt) and X_{c}=cos(nω′t) or sin(nω′t) where ω′=ω+δω and δω is the frequency error. The update equation used at 60 in FIG. 3 in this example can be written as: aₙ₊₁=aₙ+μ\overline{eX}_{c} where \overline{eX}_{c} is the averaged error product term 56.

The disclosed example takes advantage of the fact that the two requirements for updating an adaptive filter in an active noise control system (i.e, (1) updating the adaptive filter coefficients based on error magnitude and (2) separating out different engine orders using a notch filter based on the orthogonality of the tones) are independent. The conventional technique of addressing both requirements using a single convergence factor parameter within a LMS update equation has proven difficult and provides the possibility for instabilities or slow convergence. The disclosed example separates out these two functions and performs error signal multiplication (i.e., orthogonal cancellation) and LMS update in a cascaded manner. Multiplying the error signal by the averaging parameter and determining an average of the multiplied error signal in a first step takes advantage of the orthogonality of the tones to be separated out from the error signal. This provides a more tonal update term and reduces fluctuations in the error signal.

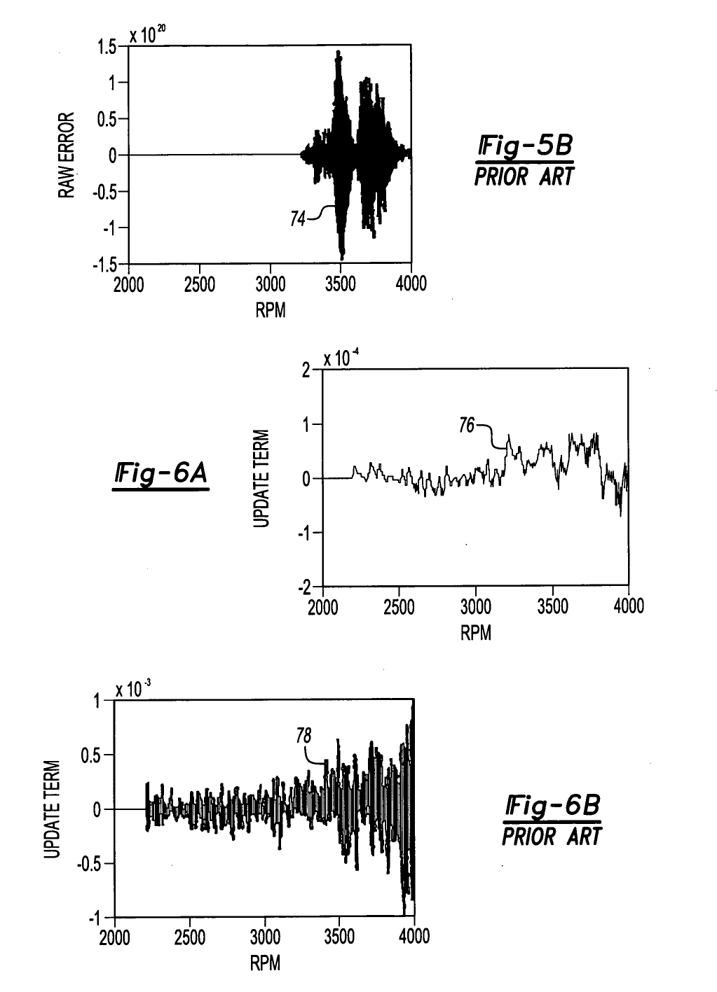

FIG. 4 graphically illustrates engine noise 70 in one example. FIG. 5A graphically illustrates raw error 72 when using a high LMS convergence factor μ after determining an average of the feedback error signal using an averaging parameter τ_{E} as described above. FIG. 5B, by contrast, shows the raw error using a conventional approach with the same μ value as that used in FIG. 5A (note the difference in scale between FIGS. 5A and 5B). The raw error 74 of FIG. 5B demonstrates instability in the system as the error approaches infinity in the range of 3500 rpm's. Comparing FIGS. 5A and 5B shows that averaging the error product e_{c} prevents instabilities in the system, allows for faster convergence and provides good cancellation

FIG. 6A graphically shows an error product term 76 having an enhanced DC component resulting from determining an average of the error signal using the disclosed techniques. By contrast, FIG. 6B shows an error product term 78 using conventional techniques. As can be appreciated from FIG. 6B, there is a significant amount of jitter in the error product term 78, which is caused by the undesired components of the error signal. Conventional LMS techniques are not able to completely filter out such jitter. Additionally, the results shown in FIG. 6A show that the disclosed example reduces the periodic variation within the frequency of interest.

The disclosed example provides the ability to better handle error signals for faster and more stable adaptive filter updating in an active noise control system. By separating out the filtering of the error signal from updating the adaptive filter coefficients, which are based on the error magnitude, the disclosed example provides a more stable system with better performance.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

I claim:

1. A method of processing an error signal in an active noise control system, comprising:
determining an average value of the error signal; and
updating an adaptive filter value using the determined average error signal value.

2. The method of claim 1, including using a least mean square update equation for updating the filter value.

3. The method of claim 2, including selecting a convergence factor that minimizes a number of cycles of using the least mean square update equation.

4. The method of claim 3, including selecting an averaging parameter for determining the average value independent of the convergence factor.

5. The method of claim 1, including selecting an averaging parameter for determining the average value based upon a lowest significant frequency.

6. The method of claim 5, including changing the averaging parameter responsive to a change in the lowest significant frequency.

7. The method of claim 5, including selecting the averaging parameter for capturing between about one and about two half-cycles of the lowest significant frequency.

8. The method of claim 5, including selecting the averaging parameter by dividing the lowest significant frequency by a sampling frequency.

9. The method of claim 8, including selecting the sampling frequency based upon a bandwidth of the noise control system.

10. An active noise control system, comprising:
a controller that determines an average value of an error signal and then updates an adaptive filter value using the determined average error signal.

11. The system of claim 10, wherein the controller uses a least mean square update equation for updating the filter value.

12. The system of claim 10, wherein the controller uses a convergence factor that minimizes a number of cycles of using the least mean square update equation.

13. The system of claim 12, wherein the controller uses an averaging parameter for determining the average value independent of the convergence factor.

14. The system of claim 10, wherein the controller uses an averaging parameter for determining the average value and wherein the averaging parameter is based upon a lowest significant frequency.

15. The system of claim 14, wherein the controller determines if the lowest significant frequency changes and changes the averaging parameter responsive to a change in the lowest significant frequency.

16. The system of claim 14, wherein the averaging parameter captures between about one and about two half-cycles of the lowest significant frequency.

17. The system of claim 14, wherein the controller determines the averaging parameter by dividing the lowest significant frequency by a sampling frequency.

18. The system of claim 17, wherein the sampling frequency is based upon a bandwidth of the system.

19. The system of claim 10, wherein the controller uses single pole averaging to determine the average error signal value.

20. A method of updating an adaptive filter value in an active noise control system comprising:
obtaining a relevant component of an error signal separately from updating the adaptive filter value based upon the error signal magnitude.
